(19) United States
(12) Patent Application Publication
(10) Pub. No.: US 2017/0087320 A1
(43) Pub. Date: Mar. 30, 2017

(54) VENTILATION WITH A VIEW MASK

(71) Applicant: Sajith Chakithandy, Al Waab (QA)

(72) Inventor: Sajith Chakithandy, Al Waab (QA)

(21) Appl. No.: 15/252,381

(22) Filed: Aug. 31, 2016

(30) Foreign Application Priority Data
Sep. 30, 2015 (QA) 20150900423

(51) Int. Cl. A61M 16/06; A61B 1/267; A61B 1/00; A61M 16/01; A61B 1/05; A61B 1/015; A61M 16/04; A61M 16/08

(52) U.S. Cl. CPC A61M 16/06; A61M 16/0488; A61B 1/2673; A61M 16/0816; A61M 16/01; A61B 1/05; A61B 1/015; A61B 1/00045; A61B 1/00195; A61B 1/00066; A61M 2230/43

(57) ABSTRACT

A facemask to abolish “difficult mask ventilation” in unconscious patients by viewing the wind pipe while delivering oxygen and incorporating channels on the facemask is described. The facemask makes it possible to oxygenate/ventilate an unconscious patient while securing a definitive airway by endotracheal intubation. The anesthetic facemask incorporates channels through which an ‘airway-slider’ fitted with a camera is inserted inside the patient's mouth, when the patient is unconscious. The airway-slider simplifies maneuvering the endotracheal tube into the patient's windpipe thus achieving a definitive clear airway with the use of just one device as opposed to the practice of needing multiple devices for the same in current anesthetic practice.

### CROSS REFERENCE TO RELATED APPLICATION

This application claims priority to QA/2015/09/00423 filed on Sep. 30, 2015, which is hereby incorporated by reference in its entirety.

### FIELD OF THE DISCLOSURE

The present disclosure relates to a facemask for ventilation and intubation of an unconscious person; and more particularly, relates to an anesthetic facemask designed to achieve effective ventilation and simultaneously help secure the airway by passing an endotracheal tube without needing to stop the ventilation.

### BACKGROUND OF THE DISCLOSURE

Mask ventilation and endotracheal intubation are fundamental aspects of airway management. The standard facemasks currently available for a rescuer or anesthetist attempting to perform the basic function of ventilation by using a rigid facemask affixed tightly on the person's face with the rescuers hands. After adequate ventilation is achieved by the facemask, an endotracheal tube is passed into the trachea by removing the facemask and using an instrument called laryngoscope.

A person who has become unconscious such as from accidental injury or medical reasons or medication typically requires a skilled medical personnel to provide the basic function of breathing. There are various procedures and mechanical devices that provide ventilation and protection against airway collapse, air leakage and aspiration for people in these situations. In resuscitation, and for minor procedures under anesthesia, a facemask is often sufficient to achieve adequate ventilation. Airway patency of the unconscious patient is maintained either by manipulation of the jaw or by the use of the nasopharyngeal or oropharyngeal airway. These are designed to provide a passage for air and oxygen to the windpipe through the nose, mouth and throat of the patient.

Difficult mask ventilation (DMV) is the term used to denote the state where ventilation of an unconscious/anesthetized person is difficult with a facemask. This situation can arise in up to 5% of all anesthesia delivered. DMV develops because of multiple factors that are technique related and/or airway related. Obesity, age older than 55 years, history of snoring, lack of teeth, and the presence of a beard are all independent predictors of DMV. DMV can be even more challenging in infants and children, because they develop low oxygen levels much faster than adults. Thus, clinicians should be familiar with the corrective measures and management options when faced with a challenging, difficult, or impossible mask ventilation situation. “Difficult intubation” is a related situation where passing an endotracheal tube into the patient's trachea is difficult or impossible with direct conventional laryngoscopy. Difficulty in achieving tracheal intubation usually occurs because of the inability to bring the vocal cords into view through the laryngoscope. This situation needs specialized equipment to solve the problem. The incidence of this situation is about 0.3 to 1.5% in the general population. Fiber optic technology and video laryngoscopy are some of the specialized equipment developed to assist with “difficult intubation” situations.

Difficulty or failure to manage the airway is the major factors underlying morbidity and mortality related to anesthesia. The ability to ventilate a patient by viewing his/her vocal cords through the airway and continuing to do so while intubation is being done would be an ideal solution in airway management. The anesthetic facemask described in the present disclosure was developed to facilitate the management of the difficult airway and to reduce the incidence of severe adverse outcomes during airway management.

While the prior art has provided current airway adjuvants for difficult mask ventilation, these efforts are blind techniques, e.g. guedal airway, the size of which is chosen by guess-estimating the distance from teeth to oropharynx. Further there are certain disadvantages of the existing prior art solutions, such as: none of the prior art solutions provide an efficient way of carrying out ventilation and intubation simultaneously with the ability to separate the facemask from the endotracheal tube once it is positioned in the windpipe; extensive set up is required for management of a difficult airway to achieve visualization of the airway, thus it is less time efficient; patients get very anxious going through these procedures before they are anaesthetized; all of the existing techniques or methods are blind techniques; and thus, will result in higher failure rates and tissue injuries to the patient; the facemask and airway coming together as described in the prior art solutions require one pipe inserted at a time, making it difficult to maintain the airway in-between manipulations.

In contrast to the prior art solutions, the present disclosure provides specific, functional design, shape & dimensions, hence removing all of the aforementioned concerns & dangers for the patient, as described herein. Some of the advantages and objects of the unique functional configuration & design of the facemask of the present disclosure are listed as follows: provides ventilation and intubation simultaneously; the facemask is easy to apply to the patient's mouth due to the grip of the wings and the channels; provides an easy and clear view of the vocal cords through camera/prism, lenses and mirrors on the airway-slider; the presence of the airway-slider with camera/prism, lenses and mirrors helps in correct placement of endotracheal tube; the facemask of the present disclosure is more comfortable to the patient as it needs less manipulation and positioning compared to conventional methods; minimal manipulation of a ventilation mask for intubation allows for reduced tissue damage; simultaneous intubation and ventilation makes the present disclosure safe for the patient and cost efficient; the facemask of the present disclosure is available in different sizes for all age groups, i.e., from infants to adults.

In one embodiment of the present disclosure, the whole set of facemask and airway slider incorporating camera/prisms, lenses and mirrors is made of disposable materials.

### BRIEF DESCRIPTION OF THE DISCLOSURE

The present disclosure seeks to improve airway management in conscious and unconscious patients through the use of a facemask capable of providing ventilation of the patient while allowing intubation of the patient simultaneously, under visual confirmation via a camera/system of lenses, prisms and mirrors. A specially designed airway-slider with an incorporated camera helps with keeping the airway patent and the vocal cords in view after being inserted through the above facemask.

It is an object of the present disclosure to provide an anesthetic facemask, which can be used to ventilate and intubate a patient at the same time. It is yet another object of the present disclosure to provide an airway-slider with a camera inserted through the facemask to view the vocal cords for proper ventilation and intubation.

It is yet another object of the present disclosure to provide two channels attachable to the facemask, thus providing a passage for the airway-slider stick and endotracheal tube while keeping the mouth open.

It is yet another object of the present disclosure to provide an airtight seal around the channels and lips in such a way that the facemask can be separated from the endotracheal tube and airway slider easily without dislodging the endotracheal tube.

It is yet another object of the present disclosure to provide an airtight seal at the lips and nose by providing “wings” on the channels and a cushioned base for the nasal part of the facemask.

It is yet another object of the present disclosure to provide a patent airway in unconscious patients by the airway-slider having a channel in its body.

It is yet another object of the present disclosure to provide a reusable monitor screen being connected to the airway slider to view the image clearly.

### BRIEF DESCRIPTION OF THE DRAWINGS

The disclosure will now be described solely by way of example and with reference to the accompanying drawings in which:

FIG. 1: shows a frontal view of an anesthetic facemask according to the present disclosure.

FIG. 2: shows a left side perspective view of the facemask according to the present disclosure.

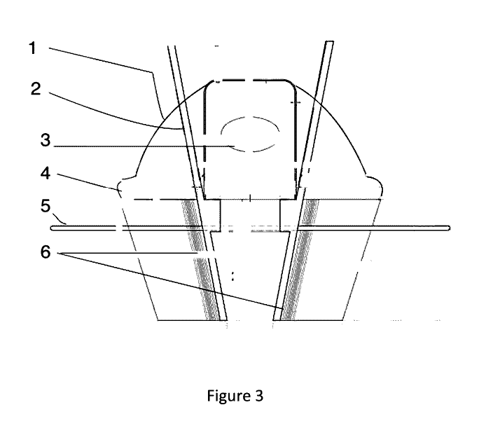

FIG. 3: shows a cut section view of the facemask showing channels.

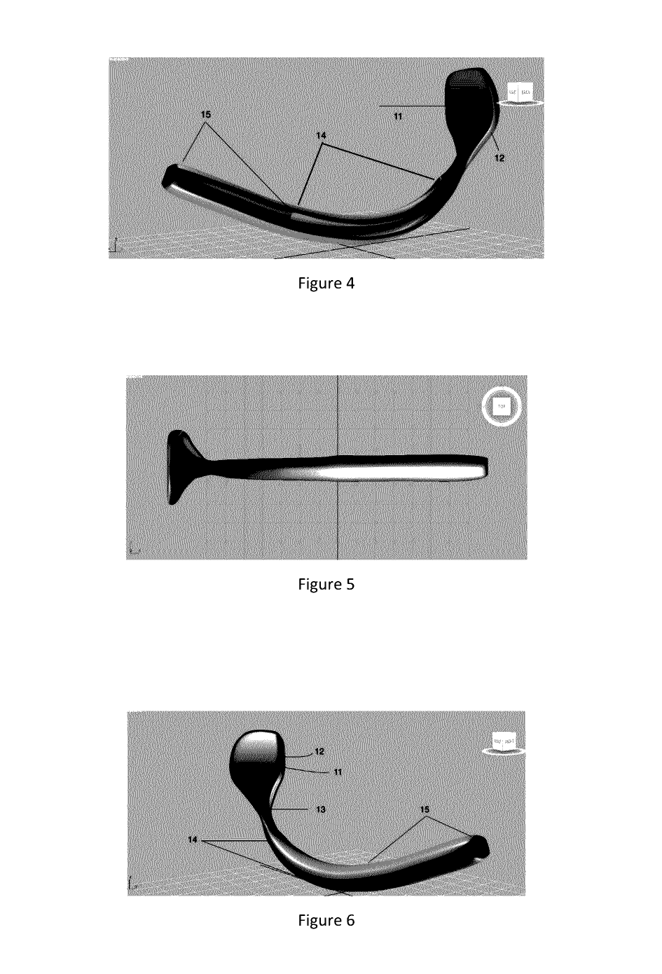

FIGS. 4 to 6: show an airway-slider with spoon shaped distal end and channel incorporated in the body.

FIG. 7: shows an inside view of the mask while inserted in a mouth.

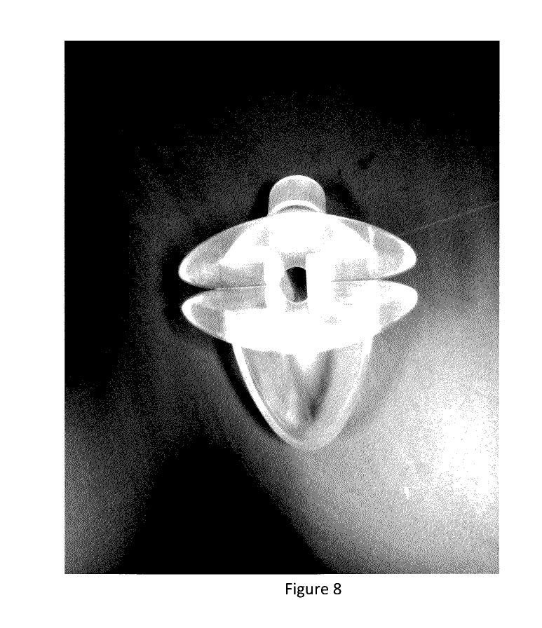

FIGS. 8 and 9: show the working prototype of the face mask.

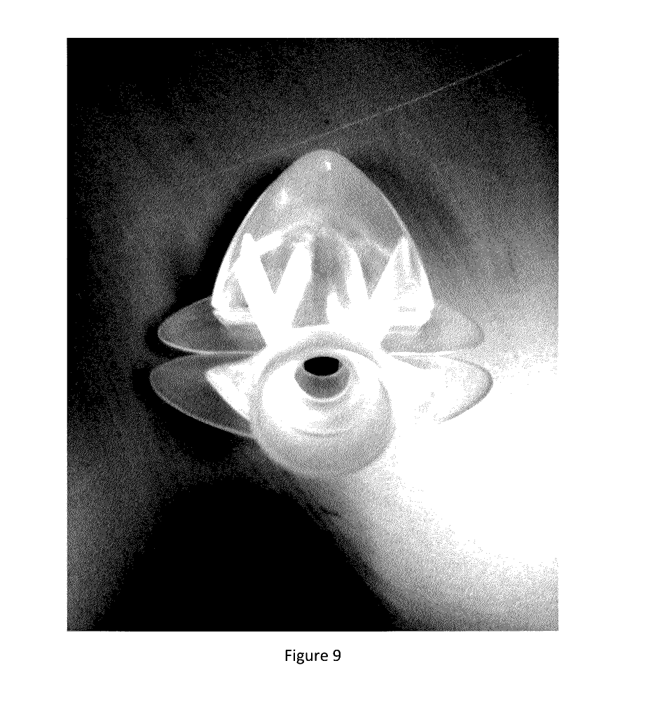

The appended claims particularly point and distinctly claim the subject matter of the present disclosure. The various objects, advantages and novel features of this disclosure will be more fully apparent from a reading of the following detailed description in conjunction with the accompanying drawings in which reference numerals refer to like parts.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

The facemask of the present disclosure is designed to be used in routine airway management during anesthesia, intensive care and in emergency medicine but will be particularly useful in “difficult airway” scenarios in clinical practice by its ability to maintain a clear airway with minimal manipulations by the operator.

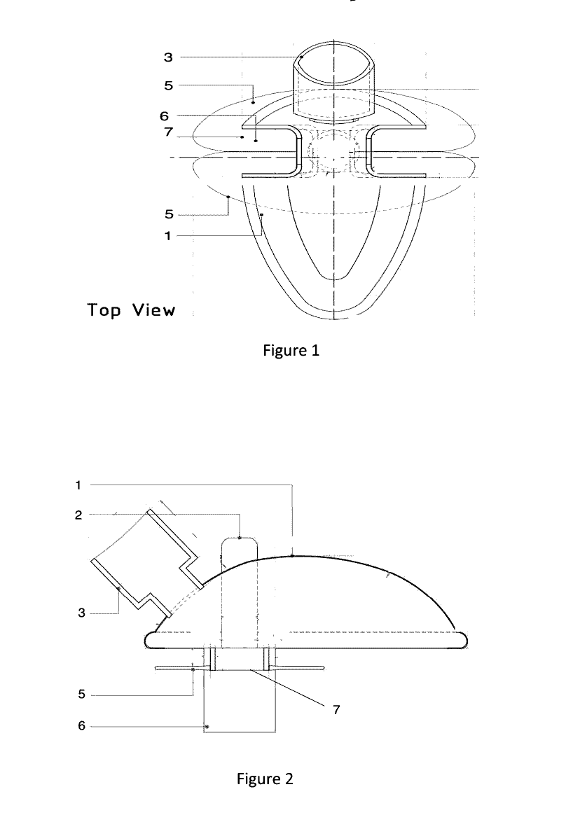

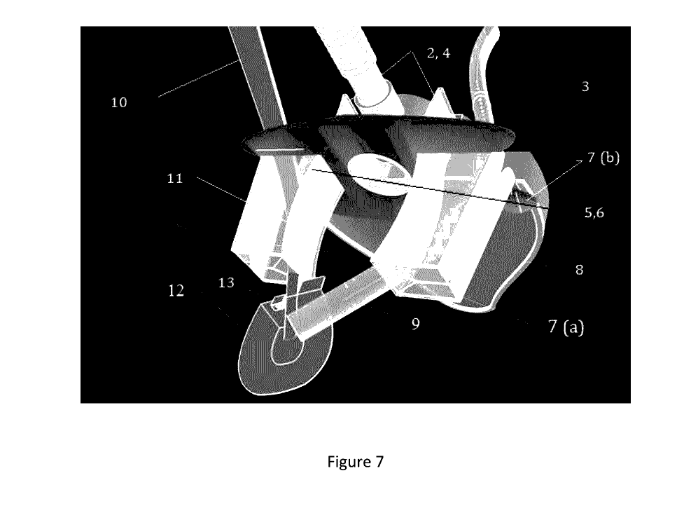

The present disclosure provides an anesthetic facemask to ventilate and intubate an unconscious person simultaneously and does not require any additional equipment. The ventilation with a view mask (FIGS. 1,2,3) incorporating two channels (6) through one of which an “airway-slider” (FIGS. 4,5,6) is fitted with a camera (13) is inserted inside the mouth when the patient is unconscious. The airway-slider simplifies maneuvering an endotracheal tube (9) inserted via the other channel (6) in to the patients' windpipe.

The present disclosure describes an assembly of facemask (1) with ventilation port (3) and channels (6) with wings (5, 7). The channels are integrated to the lower end of the mask and are inserted in patient's mouth.

More specifically, the present disclosure consists of the following parts:

Channels (6): help to keep the mouth open when it is inserted inside the mouth. Also it holds the mask in place along with the wings (5,7) attached to it, the wings on the channels when positioned in between the lips and gum/teeth of the patient keep the facemask in position on the patients face, thus freeing operator hands. The channel extends up to the front ⅓rd of the tongue from each side of the ventilation port (3). It is ‘C shaped’ or ‘O shaped’ in cross section thus creating space on both sides to insert an airway-slider (FIGS. 4,5,6) on the right side and an endotracheal tube (9) on the left side or vice versa.

Wings on Channels (7(a) and 7(b)): acts as a valve by staying in between the channels (6) and inside of the cheeks and on the teeth and gums when inserted properly, preventing air leak.

Inside part of the channel (left side/right side): C shaped/O shaped in cross section it extends from the front of the mask to reach inside the mouth near the tip of the tongue. It is with or without wings creating an airtight seal between the cheeks and the facemask. One of the channels allows the airway-slider to be inserted through it and engage with the endotracheal tube inserted through the other channel.

Airway-slider (FIGS. 4, 5, 6): this is in effect approximately 30 cm long, approximately L shaped to conform to airway anatomy, and it has a camera at its slide shaped tip which helps to view inside the mouth after being passed inside the mouth through the right sided channel of the mask. The inner end of the airway-slider is shaped like a spoon with a design feature helping it to engage an endotracheal tube inserted from the left side of the mouth. The camera is positioned a little away from the end giving adequate visualization of the vocal cords. The function of this device is twofold: first, to act as an oral airway inside the mouth, thus allowing ventilation of the patient with a view of the vocal cords maintained; and second, to help slide the endotracheal tube into the wind pipe by minimal maneuvering inside the mouth. The airway-slider may be fixed at any position or angle on the ventilation facemask by use of magnets or Velcro, thus achieving a clear airway and also guide the endotracheal tube into patients' wind pipe without having to stop ventilation of the patient.

Ventilation port (3): attached at the lower end of the facemask in line with the nasal opening. It connects to a standard anesthetic circuit or any other breathing circuit. This port is in the center bottom part of the facemask with a clear shoulder part connecting it to the rest of the facemask.

Nasal mask (8): this is the part covering the nose and is attached to the channels at an elevation of 90 degrees this creating an airtight seal.

With specific reference to the drawings in detail, it is stressed that the particulars are shown for purposes of illustrative discussion of the preferred embodiments of the present disclosure only, and are presented in the cause of providing what is believed to be the most useful and readily understood description of the principles and conceptual aspects of the disclosure. In this regard, no attempt is made to show structural details of the disclosure in more detail than is necessary for a fundamental understanding of the disclosure, the description taken with the drawings making apparent to those skilled in the art how the several forms of the disclosure may be embodied in practice.

In one embodiment of the present disclosure, a facemask for ventilation and intubation of the patient comprising a mask with two channels opening into the mouth and having wings on the channels to prevent air leak when placed on the patient's face and oxygen and anesthetic gases are delivered to the patient through its ventilation port.

In another embodiment, an airway-slider for maintaining a clear airway and maneuvering an endotracheal tube into the wind pipe is inserted through the channels on the facemask above and help to keep the airway clear for ventilation by viewing the vocal cords with the camera on it.

In yet another embodiment of the present disclosure, an endotracheal tube is inserted through the channel on the left and an airway-slider is inserted through the channel on the right of the mask into the mouth of a patient. The endotracheal tube is then maneuvered into the wind pipe with the airway-slider under vision.

In yet another embodiment of the present disclosure, the endotracheal tube will be maneuvered through the ventilation port into a patient's nose and guided into the windpipe with the airway-slider in the throat.

In yet another embodiment of the present disclosure, the airway-slider comprising three parts:

Part 1 (Pharyngeal port) (11): has a thin neck angled about 30 to 50 degrees to a spoon shaped end incorporating a camera and has a ridge shaped edge (12) to guide the endotracheal tube;

Part 2 (body) (14): has a hollow center open on one side and is in a smooth curve corresponding to oral cavity; and

Part 3 (handle) (15) continuation of the body and it has a wired connection port to be connected to the reusable monitor.

In yet another embodiment of the present disclosure, the airway-slider is incorporated with a camera at its inner end, which provides a video of the inside of the patient on a monitor screen at the bedside.

In yet another embodiment of the present disclosure, the facemask is used to clear the airway and achieve endotracheal intubation without having to stop ventilation to the patient.

In yet another embodiment of the present disclosure, the facemask is used for all age groups of patients by manufacturing them in five different sizes.

In yet another embodiment of the present disclosure, the facemask with incorporated channels will also serve as a soothing dummy to small children needing pre-oxygenation before getting them to sleep by using a softer material for the channels.

In yet another embodiment of the present disclosure, the airway-slider may be designed with an extra channel on it for providing oxygen or using suction near the vocal cords under vision. The ventilator port on the facemask will not be needed in this situation.

In yet another embodiment of the present disclosure, the channels are designed separately from the mask so that they can be used separately or together as needed.

In yet another embodiment of the present disclosure, the facemask is of ‘Q’ shaped and hence may be termed as Q-mask.

What is claimed is:

1. A facemask (1) for ventilation and intubation of a patient comprising:
a nasal mask (8) and two channels (6) with or without wings (5,7) for use of ventilating a patient without air leak, wherein the nasal mask is designed as the main assembly with a single ventilation port (3) at one end delivering air and oxygen and two channels on either side to provide access to the patient's mouth;
an airway-slider incorporating a camera (13) which can be inserted via the channels (6) for the purpose of maintaining the airway by clear visualization of the vocal cords and also to guide an endotracheal tube (9) into the trachea.

2. The facemask as claimed in claim 1 wherein an endotracheal tube is inserted through channel (6) on a first side and the airway-slider is inserted through channel (6) on an opposing second side of the mask.

3. The facemask as claimed in claim 1 wherein the channels (6) are ‘C’ or ‘O’ shaped and/or the wings (5,7) being molded thereto separate from nasal mask (8) and an operator can attach both of which onto the mask with a linking mechanism.

4. The facemask as claimed in claim 1 wherein the channels (6) have two channel handles (2, 4) for better gripping of the face mask.

5. The facemask as claimed in claim 1 wherein a ventilation port (3) is designed either as a male or female port at the lower end of the nasal mask (8) for connection to a standard anesthetic circuit.

6. The facemask as claimed in claim 1 being connected to a source of at least one of oxygen, air, and anesthetic vapor via the ventilation port and the expired gases from the patient being monitored for their contents to achieve adequate oxygenation, ventilation and anesthesia for the patient.

7. The facemask as claimed in claim 1 being used to maintain a clear airway for the patient at all times by visualizing the vocal cords of the patient and fixing the airway slider at any position or angle onto the ventilation mask by use of magnets or Velcro thus achieving a clear airway and capable of guiding the endotracheal tube into the patient's wind pipe without having to stop ventilation of the patient.

8. The facemask as claimed in claim 1 wherein the facemask is fabricated from a material selected from the group consisting of thermoplastic material, silicone or elastomer.

9. An airway-slider (10) for use in the facemask of claim 1 wherein said airway-slider further comprises three parts:
a) Pharyngeal part (11) having a thin neck angled from about 30 degrees to about 50 degrees to a body (14) and having a slide shaped distal end incorporating the camera (13) or system of mirrors, prisms and lenses;
b) Body (14) having a hollow center open on a first side and being c-shaped in cross section, designed as a smooth curve corresponding to the oral cavity of a patient and being a channel for administering oxygen or suction;
c) Handle (15) being a continuation of the body (14) and having a wired connection port to be connected to the reusable video monitor.

10. The airway-slider as claimed in claim 9 comprising the camera (13) at its inner end which provides a video of the inside of a patient's mouth on a monitor screen at the bedside.

11. The airway slider (10) as claimed in claim 9 comprising a system of mirrors, lenses and prisms to transmit the images from inside a patient's mouth to an operator eyepiece on the handle of airway slider.

12. The airway slider as in claim 9 comprising a Velcro or magnetic system incorporated on its handle so as to fix it at any specific position on to the c-shaped channel in the mask.

13. The airway-slider as claimed in claim 9 wherein the airway-slider is made of a material such as a thermoplastic or glass.
